(19) United States
(12) Patent Application Publication
(10) Pub. No.: US 2024/0165356 A1
(43) Pub. Date: May 23, 2024

(54) RESPIRATOR MASK WITH PHYSIOLOGICAL MONITORING SYSTEM

(71) Applicant: 3M INNOVATIVE PROPERTIES COMPANY, St. Paul, MN (US)

(72) Inventors: Darin K. Thompson, Huntersville, NC (US); Richard J. Sabacinski, Charlotte, NC (US); Eric J. Bassani, Denver, NC (US); Jeremy V. Barbee, Oakboro, NC (US)

(21) Appl. No.: 18/286,704

(22) PCT Filed: May 5, 2022
(86) PCT No.: PCT/IB2022/054169

§ 371 Date: Oct. 12, 2023

Related U.S. Application Data
(60) Provisional application No. 63/185,030, filed on May 6, 2021.

(51) Int. Cl. A61M 16/06; A61B 5/00; A61B 5/0205; A61B 5/024; A62B 7/00; A62B 9/00; A62B 18/02; A62B 18/08; H04R 1/02; H04R 1/10

(52) U.S. Cl. CPC A61M 16/06; A61B 5/02055; A61B 5/6803; A61B 5/746; A62B 7/00; A62B 9/006; A62B 18/02; A62B 18/08; H04R 1/028; H04R 1/1008; A61B 5/02427; H04R 2460/13

(57) ABSTRACT

The present invention relates to a self-contained breathing apparatus (SCBA) facemask with a facemask-resident physiological monitoring system including at least one integrated physiological sensing unit with at least one physiological sensor. The face-mask may also comprise an integrated bone conduction communication system. In an embodiment, the physiological sensor comprises a contact temperature sensor to monitor body temperature of the user. In another embodiment, the physiological sensing sensor comprises photoplethysmographic sensor to monitor heart rate and respiration of the user. The present inventions also relates to a method of performing physiological monitoring of a user of an SCBA facemask and communicating sound to the user of the SCBA facemask.

### BACKGROUND

A self-contained breathing apparatus (SCBA) is an apparatus generally used to provide respiratory protection to a person that may be entering an objectionable, oxygen-deficient, and/or otherwise potentially unbreathable or toxic environment. Such apparatus are often worn by firefighters, first responders, and so on, sometimes in conditions of extreme heat and/or humidity, and/or while performing activities involving significant physical exertion.

### SUMMARY

In broad summary, herein is disclosed a self-contained breathing apparatus (SCBA) facemask comprising a facemask-resident physiological monitoring system comprising at least one integrated physiological sensing unit that comprises at least one physiological sensor. The facemask may also comprise an integrated bone conduction communication system. These and other aspects will be apparent from the detailed description below. In no event, however, should this broad summary be construed to limit the claimable subject matter, whether such subject matter is presented in claims in the application as initially filed or in claims that are amended or otherwise presented in prosecution.

### BRIEF DESCRIPTION OF THE DRAWINGS

FIG. 1 is a partially exploded view of an exemplary SCBA facemask comprising at least one integrated physiological sensing unit.

FIG. 2 is a side perspective view of an exemplary SCBA facemask comprising at least one integrated physiological sensing unit.

FIG. 3 is a rear perspective view of an exemplary SCBA facemask comprising at least one integrated physiological sensing unit.

FIG. 4 is a rear perspective view of another exemplary SCBA facemask comprising at least one integrated physiological sensing unit.

FIG. 5 is a partially exploded view of another exemplary SCBA facemask comprising at least one integrated physiological sensing unit.

FIG. 6 is an isolated rear perspective view of an exemplary integrated physiological sensing unit of an SCBA facemask.

FIG. 7 is an isolated rear perspective view of an exemplary integrated physiological sensing unit of an SCBA facemask.

FIG. 8 is an isolated rear perspective view of an exemplary integrated physiological sensing unit of an SCBA facemask.

Like reference numbers in the various figures indicate like elements. Some elements may be present in identical or equivalent multiples; in such cases only one or more representative elements may be designated by a reference number but it will be understood that such reference numbers apply to all such identical elements. Unless otherwise indicated, all figures and drawings are not to scale and are chosen for the purpose of illustrating different embodiments of the invention. In particular the dimensions of the various components are depicted in illustrative terms only, and no relationship between the dimensions of the various components should be inferred from the drawings, unless so indicated. Although terms such as “first” and “second” may be used in this disclosure, it should be understood that those terms are used in their relative sense only unless otherwise noted.

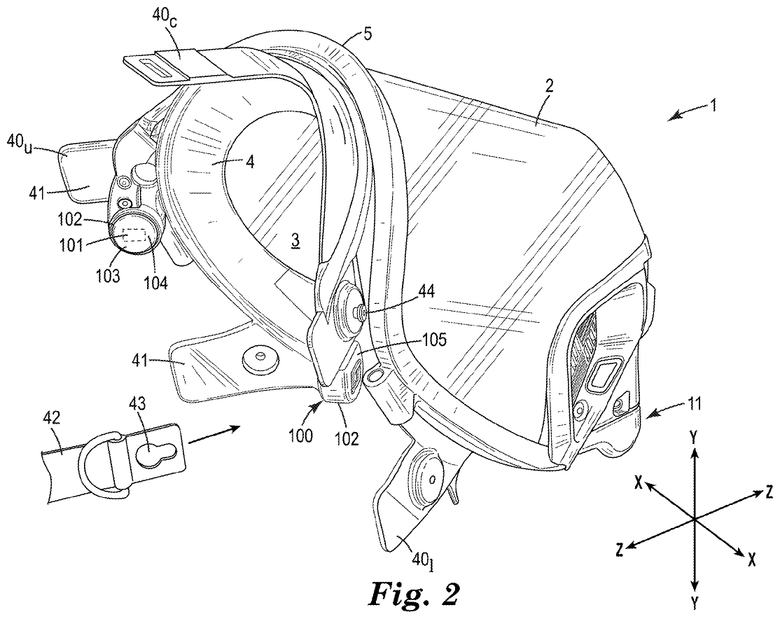

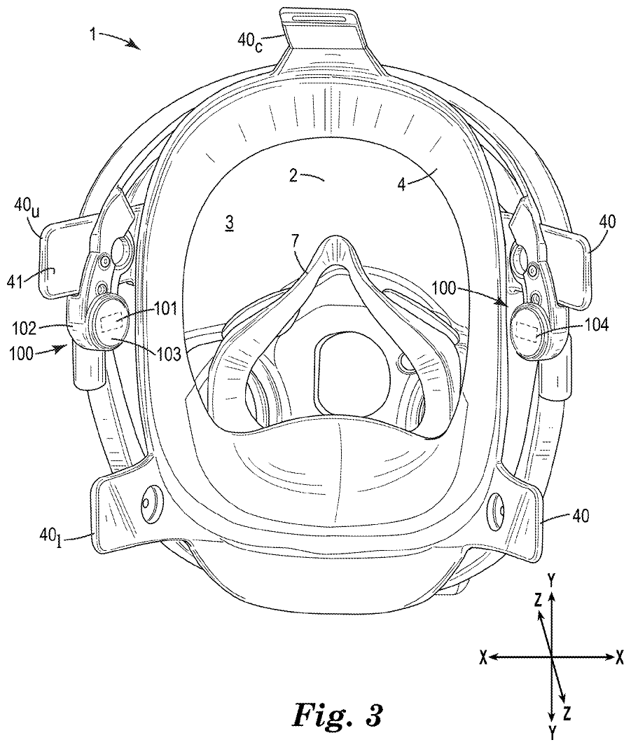

As used herein, geometric and positional parameters will be used with reference to a facemask, regulator, and components thereof, as positioned on the face of an upright human user. In this context, terms such as forward and front refer to a direction generally away from the user's face, and rear, rearward, and so on, refer to a direction generally toward the user's face. Thus for example, with respect to the exemplary facemask shown in FIG. 2, forward is to the right on the page as viewed and rearward is to the left on the page as viewed. The vertical direction, and terms such as up, down, above, below, etc., will have their normal meaning with reference to a facemask that is positioned on the face of an upright human user. The lateral direction and terms such as left, right, etc., will have their normal meaning with reference to such a facemask. For reference, FIGS. 2 and 3 are provided with axes, with the x axis indicating the lateral direction, the y axis indicating the vertical direction, and the z axis indicating the forward-rearward direction. Terms such as inward and outward are used with respect to items that exhibit a particular orientation with regard to the user's head; inward signifies a direction toward the head and outward signifies a direction away from the head.

The term “configured to” and like terms is at least as restrictive as the term “adapted to”, and requires actual design intention to perform the specified function rather than mere capability of performing such a function. All references herein to numerical values (e.g. dimensions, ratios, and so on), unless otherwise noted, are understood to be calculable as average values derived from an appropriate number of measurements.

### DETAILED DESCRIPTION

Facemask

Disclosed herein is a facemask 1 that may be used as part of a self-contained breathing apparatus (SCBA) arranged to deliver breathable air to a human user of the apparatus. Such an SCBA may comprises one or more tanks comprising a high-pressure breathable gas, mounted on a harness so that the tank(s) can be comfortably supported e.g. on the back of the user. The SCBA will comprise associated hoses and equipment so that breathable air can be supplied to the facemask. In many instances, this equipment may include a first-stage regulator that reduces the pressure of the breathable air from the tank pressure to an intermediate pressure. The breathable air at this intermediate pressure is then delivered to a second-stage regulator that, in many embodiments, may be attached to facemask 1. Such a regulator can further reduce the pressure of the air to a suitable value (e.g. to near-atmospheric pressure) and deliver the air to facemask 1. SCBAs, regulators and facemasks for SCBAs, and so on, are described e.g. in U.S. Pat. No. 4,269,216; in U.S. Provisional Patent Application 62/879,279 and in the resulting PCT application published as WO 2021/019348; and, in U.S. Provisional Patent Application 62/745,154 and in the resulting PCT application published as WO 2020/075045, all of which are incorporated by reference in their entirety herein.

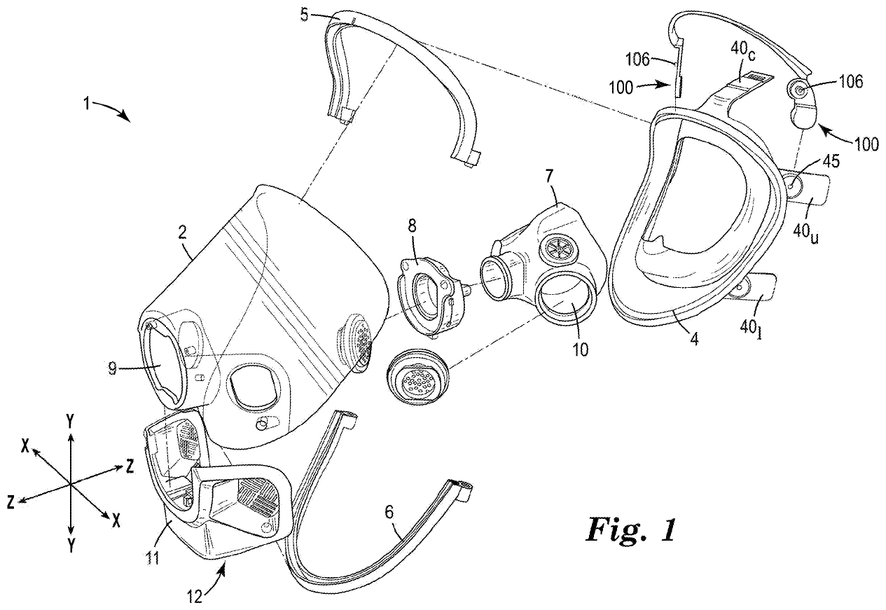

An exemplary facemask 1 is shown in partially exploded view in FIG. 1 and in side view and rear view in FIGS. 2 and 3. Any such facemask will comprise a generally forward-facing clear pane or lens 2 through which the user can see, and will define an interior volume (air space) 3 when fitted to the face of the user. Such a face mask will comprise a face seal 4 (and in some embodiments may further comprise upper and lower seals 5 and 6), made of a compliant material such as silicone, EPDM, polyisoprene, or the like, that ensures that the face seal is conformable to the user's face in a manner that minimizes or eliminates any air leaks. In many embodiments a face seal 4 may take the form of a single, integrally molded piece of compliant material as evident in FIG. 1; in some embodiments such one or more strap coupling elements (described below) may be integrally molded with the face seal. Exemplary facemask 1 comprises a nosecup 7 that resides within interior volume 3 and that fits about the nose and mouth of the user to deliver breathable air thereto. Nosecup 7 will often be made of a compliant material in similar manner to face seal 4.

A facemask 1 will comprise a coupler 8 (e.g. a connection or fitting) that allows a regulator or like device to be mounted on, and fluidly connected to, the facemask, so that the regulator can deliver breathable air to the facemask. A suitable regulator (not shown) may access coupler 8 via an access opening 9 provided e.g. in the forward end of pane 2. In some instances, a facemask 1 may comprise various other components and accessories. For example, nosecup 7 of facemask 1 of as shown in FIG. 1 comprises side-fittings 10 to which voice amplifiers or radio direct interface devices (unnumbered) can be attached. These and other ancillary items that may be present on a facemask will not be discussed in detail herein. A facemask 1 may further comprise an electronic control unit 11, which may contain (and protect within a control unit housing) various electronic components as discussed in detail herein. In some embodiments an electronic control unit 11 may comprise a receptacle 12 that accepts one or more batteries to serve as a portable power source for various electronic components and systems of the facemask.

An SCBA facemask as disclosed herein (comprising a face seal and other components as described above) will be distinguished from other items that may be disposed on a person's head. Items that do not qualify as an SCBA facemask include (but are not limited to) eyeglasses, hats and helmets, and headbands and head suspensions (including special-purpose headbands and head suspensions that are configured for supporting one or more items such as a hardhat, an optical instrument, etc.)

Facemask 1 may comprise a number of strap coupling elements 40 as shown in exemplary embodiment in FIGS. 1-3. Such strap coupling elements are configured to each be coupled (e.g., fastened, latched, buckled, attached, etc.) to a complementary strap of a head harness. In some embodiments, a facemask 1 may comprise a first, lower pair of strap coupling elements 40ₗ that flank a lower portion of the facemask, a second, upper pair of strap coupling elements 40ᵤ that flank an upper portion of the facemask, and an additional crown strap coupling element 40_{c} at the uppermost point of the facemask, all as indicated in FIG. 3. In such an instance, a head harness that is to be used with the facemask may comprise five complementary straps that can be connected to the respective strap coupling elements. In some embodiments, a strap coupling element of a facemask may comprise a fastener (e.g. a snap-fastener at its terminal end) that is fastenable to a complementary fastener of a strap of a strap harness e.g. by snapping. In some embodiments a strap coupling element may comprise a slot that allows a terminal end of a harness strap to be passed through the slot and e.g. turned back and pulled tight to secure the harness strap to the strap coupling element. It will be appreciated that the specific way in which a harness strap is coupled to a strap coupling element of the facemask is not crucial and any system or arrangement of fasteners, buckles, latches, etc., may be used for such purposes. Exemplary head harnesses and straps, buckles, etc. thereof that may be suitable for use with a facemask 1 as disclosed herein are described e.g. in U.S. Patent Application Publication 2019/0118009 and in U.S. Pat. No. 9,603,397, both of which are incorporated by reference herein in their entirety.

The harness straps, the strap coupling elements 40 of facemask 1, or both, will be configured so that the straps and strap coupling elements can be adjustably connected to ensure a proper fit to the wearer's head. In some embodiments, strap coupling elements 40 may be attached (e.g. permanently attached) to face seal 4. In particular embodiments, one or more such strap coupling elements may be integral extensions of face seal 4 as noted above. In some embodiments, an arrangement may be used in which a strap coupling element 40 (e.g. that is a molded extension of face seal 4) has a strap coupling extension 42 attached thereto as shown in FIG. 2. Such an extension 42 may have e.g. a keyhole mounting orifice 43 that can be secured onto a complementary flanged post 44 of strap coupling element 40 so that element 40 and extension 42 combine to form a hybrid coupling element to which a harness strap may be connected. It is again emphasized that the particular attachment schemes and arrangements that are disclosed herein are exemplary and that any suitable arrangement by which straps of a head harness may be connected to a facemask 1 and adjusted (e.g. snugged tight) are encompassed by the disclosures herein.

A facemask 1 as disclosed herein will comprise a physiological monitoring system that is resident on the facemask. By this is meant that all components of the system that are needed to obtain and process data as disclosed herein, are resident on the facemask. Information that is generated by the physiological monitoring system (e.g. an alert signal as discussed later herein) can be communicated by the monitoring system to a remote device and/or to a user of the facemask. Such a mask-resident physiological monitoring system will comprise at least one physiological sensing unit 100 as shown in exemplary embodiment in FIGS. 1-3. In the illustrated embodiments, two such sensing units (a left unit and a right unit) 100 are depicted; however, in various embodiments, any number of such sensing units (e.g. one, three, four, or more) may be used.

By a physiological sensing unit 100 is meant a unit that comprises at least one physiological sensor 101 that is configured to be positioned close to (e.g., within 4, 2, or 1 mm of, often, in contact with) the head of a wearer of facemask 1, so that at least one physiological parameter of the wearer can be measured, and monitored over time. In some embodiments, a parameter that is measured may be the body temperature of the wearer. In some embodiments, a parameter that is measured may include any or all of the heart rate, respiration rate, or oxygen saturation of the wearer. In various embodiments, any combination of these and any other physiological parameters may be measured. In some embodiments, a physiological sensing unit 100 may comprise multiple physiological sensors 101, whether of the same type (i.e. configured to monitor the same physiological parameter) or of different types, configured to measure different physiological parameters. Whatever the number and type of parameters that are to be measured, any such sensing unit 100 and all sensors 101 thereof will be integrated into facemask 1. By this is meant that the sensing unit is attached to facemask 1 in such a manner that donning facemask 1 will automatically position the sensing unit, and all sensors thereof, in the proper location for obtaining the physiological data (although in some instances some fine adjustment by the wearer may be helpful for optimum performance). In other words, an integrated physiological sensing unit will not be provided as a separate unit that must be positioned on the wearer's head separately from the donning of facemask 1. It is noted however that an integrated sensing unit may, for example, be able to be separated from facemask 1 e.g. for cleaning or maintenance.

In an exemplary illustration, a physiological sensing unit 100 may comprise a physiological sensing unit housing 102 that comprises an inward surface 103 that comprises a contact area 104 that is configured to closely abut (e.g. contact) a designated area of the wearer's head and within which at least one physiological sensor 101 is located, as evident from FIGS. 2 and 3. An outward portion 105 of housing 102 (and/or a portion of an arm or strut that supports housing 102) may be in contact with an inward surface 41 of an adjacent strap coupling element 40. Such an arrangement can provide that as the strap coupling element 40 and a harness strap to which it is connected are snugged tight, this tightening will press contact area 104 of housing 102 of sensing unit 100 against the wearer's head so that the desired monitoring can be performed.

In the particular arrangement depicted in FIGS. 1-3, a sensing unit 100 may comprise a strap engagement element 106 (e.g. a post) that is configured to penetrate into and/or pass through a complementary orifice 45 of a strap coupling element 40, as most easily seen in FIG. 1. This can hold sensing unit 100 securely in place relative to facemask 1 and can ensure that sensing unit 100 is properly positioned relative to a user's head when facemask 1 is donned. (In some embodiments such a post 106 may protrude through orifice 45 so as to function as a post 44 that allows a strap, and/or a strap coupling extension, to be attached thereto as described above.) It will be appreciated that the above descriptions are exemplary and that there are many ways in which a sensing unit may be located on, and e.g. attached to, a facemask for the purposes described herein.

In some embodiments, two such physiological sensing units 100 may be provided, e.g. for each side of the wearer's head, as most easily seen in FIG. 1. In some such embodiments the sensing units may be connected to each other by an arcuate band 36, again as most clearly seen in FIG. 1. Such a band 36 is an optional component and, if present, may serve a variety of purposes. In some embodiments, such a band may be resiliently biased and/or sized so that the band urges each sensing unit 100 inward to help press the sensing units 100 firmly against the wearer's head. In some embodiments, the band may connect the two sensing units to each other to facilitate installation of the sensing units into the facemask, and/or to provide a protected pathway for communication (e.g. electrical wiring or fiber optic cabling) and/or for electrical power to be supplied to the sensing units.

In embodiments of the type depicted in FIGS. 1-3, a physiological sensing unit 100 may be positioned e.g. generally below a strap coupling element 40 (and any portion of a harness strap that may be connected to that strap coupling element), as most easily seen in FIG. 3. This may be arranged e.g. by using a sensing unit housing 102 that is vertically elongated, and/or by disposing the sensing unit housing 102 at the end of a downward-extending arm or strut.

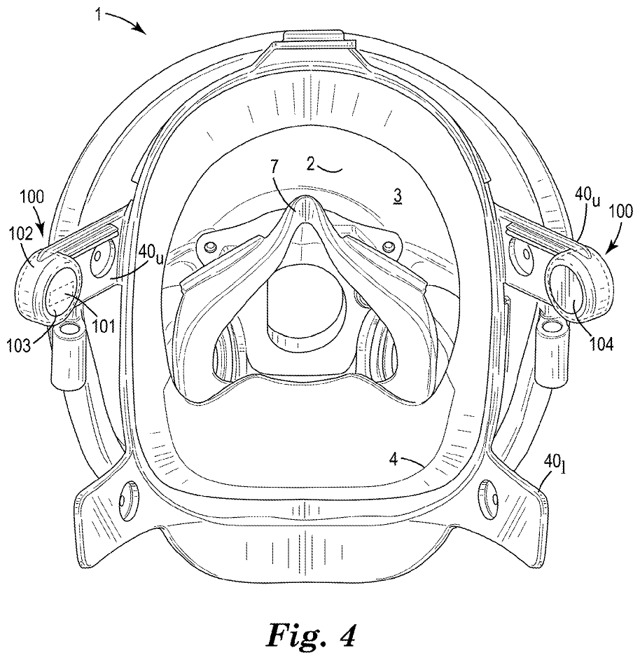

A slightly different arrangement of a physiological sensing unit 100 is depicted in FIG. 4. In such embodiments a sensing unit may be disposed more or less directly on an inward side of a strap coupling element 40. The sensing unit housing 102 may be connected or attached to the strap coupling element 40 in any suitable manner. The strap coupling element 40 may comprise e.g. any of the features or functionalities described above whereby the element can be mated with, and joined to, a strap of a harness. Such an arrangement can provide that pulling the strap coupling element 40 and its associated harness strap tight can press the sensing unit 100 against the wearer's head. However, such an arrangement is not necessarily required as long as the sensing unit can be contacted with the user's head to a sufficient degree to allow the sensing unit to function. In many embodiments this may be achieved by an arrangement of the general type depicted in FIGS. 1-3, or of the general type depicted in FIG. 4. However, it will be appreciated that there are other arrangements that can allow a sensing unit 100 to be pressed against a wearer's head when a strap coupling element is snugged tight. For example, in some embodiments a sensing unit 100 may not necessarily be directly attached to a strap coupling element (e.g., it may be attached to some other component of facemask 1) but nevertheless will be positioned (e.g. near, or inward of, the strap coupling element) so that when the strap coupling element is tightened, the sensing unit is pressed against the wearer's head. Any such arrangement that allows tightening of the strap coupling element to cause the sensing unit housing to be pressed against the user's head (whether or not the sensing unit is actually attached to the strap coupling element) will be referred to as the sensing unit being associated with the strap coupling element.

Discussions later herein will make it clear that for some purposes, it may preferable for a physiological sensing unit 100 to be positioned on facemask 1 so that when facemask 1 is donned, at least one physiological sensor 101 of sensing unit 100 will be positioned proximate (i.e., within 1 cm of) an artery that is a continuation of, or that branches from, the external carotid artery. (In this context, “branches from” includes sub-branches, sub-sub-branches, and so on.) Such arteries include the superficial temporal artery and its branches, sub-branches etc.; and, the posterior auricular artery and its branches, sub-branches and so on. To achieve such an arrangement, in some embodiments a physiological sensing unit 100 will be associated with a strap coupling element 40 that is an upper strap coupling element 40ᵤ.

In some embodiments, a sensing unit 100 may be configured to be positioned so that the contact area 104 of the sensing unit will directly contact the skin of the user's head. In some embodiments, the sensing unit may be configured to allow at least a small amount of the user's hair to be present between the user's skin and contact area 104. Such arrangements will depend on the operating mechanism of the sensor; in particular, whether or not it needs to be in direct contact with the user's skin to function adequately. It is thus noted that in this context, the terminology of “in contact with a user's head” and similar terminology encompasses not only direct contact with the skin of the user's head, but also situations in which a layer of hair is present between the sensing unit and the skin of the user's head.

A mask-resident physiological monitoring system may comprise any necessary circuitry to allow one or more physiological sensors 101 of one or more physiological sensing units 100 to be operated and to allow the data obtained therefrom to be processed. The term “circuitry” broadly encompasses any suitable electronic components needed for functioning, e.g. one or more integrated circuits, interconnections, and so on. In some embodiments at least some processing of data that is obtained by a sensor 101 may be performed by circuitry that is present in housing 102 of sensing unit 100. In some embodiments at least some such processing may occur on-board the facemask, but in a location other than within the sensing unit housing 102. For example, at least some such processing may occur within an above-mentioned electronic control unit 11. In some facemasks, an electronic control unit 11 may already be present and may comprise circuitry for purposes of e.g. communication and/or for operation of one or more devices such as e.g. a PASS device, a mask-mounted thermal imaging system, etc. In such embodiments, the electronic control unit 11 may merely need to be modified to include circuitry to operate sensing unit(s) 100. In some embodiments, communication between such an electronic control unit 11 and a sensor 101 may be wireless, e.g. by Bluetooth or any suitable short-range communication protocol. (Even if communication with a sensor 101 is wireless, in some embodiments it may still be desirable to provide a hard-wired connection in order to supply electrical power to the sensor).

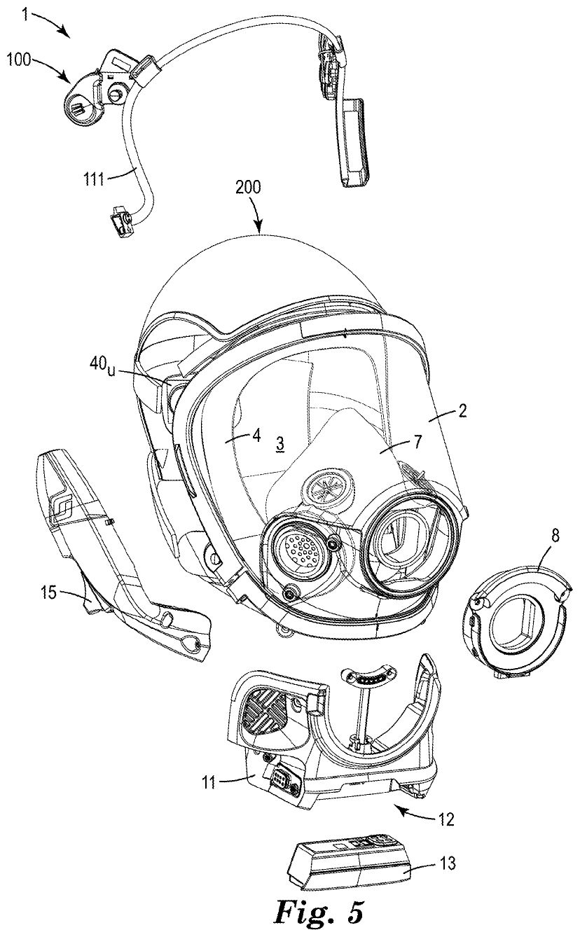

A slightly different arrangement is depicted in FIG. 5, which is a partially exploded view of another exemplary facemask 1, from a different perspective as from FIG. 1. This facemask 1 similarly comprises first and second (left and right) physiological sensing units 100, as well as the previously described pane 2, nosecup 7, coupler 8, face seal 4, etc. (In the illustrated embodiment, an exemplary head harness 200 is also depicted, the harness including a mesh cover in addition to the above-described assortment of straps.)

In the depicted embodiment of FIG. 5, facemask 1 comprises (in addition to electronic control unit 11), a sensor control module 15, which is a dedicated electronic module that comprises circuitry to operate a sensing unit 100, to receive and process data from the sensor(s) 101 of sensing unit 100, and so on. (In the absence of any such additional sensor control module 15 as a physically distinct, dedicated entity, an electronic control unit 11 will comprise circuitry that can serve as a sensor control module.) Such a sensor control module 15 may be connected to electronic control unit 11 and/or may be provided electrical power therefrom (e.g. by way of a battery 13 as indicated in FIG. 5). Or, in some embodiments, a sensor control module 15 may contain its own power source, e.g. battery. In some embodiments, a sensor control module 15 and/or an electronic control unit may draw electrical power from a power source that is located on a harness of an SCBA system rather than from a source that is resident on the facemask. Such arrangements are described, for example, in U.S. Patent Application Publication 2010/0139661, which is incorporated by reference herein in its entirety. As visible in FIG. 5, a sensing unit 100 (in the depicted arrangement, a pair of sensing units) may be provided with a hardwired electrical connection 115 by which sensor control module 15 can relay electrical power (from whatever source) to the sensing unit(s), can control the sensing units, can receive data from the sensors so that the data can be processed, and so on.

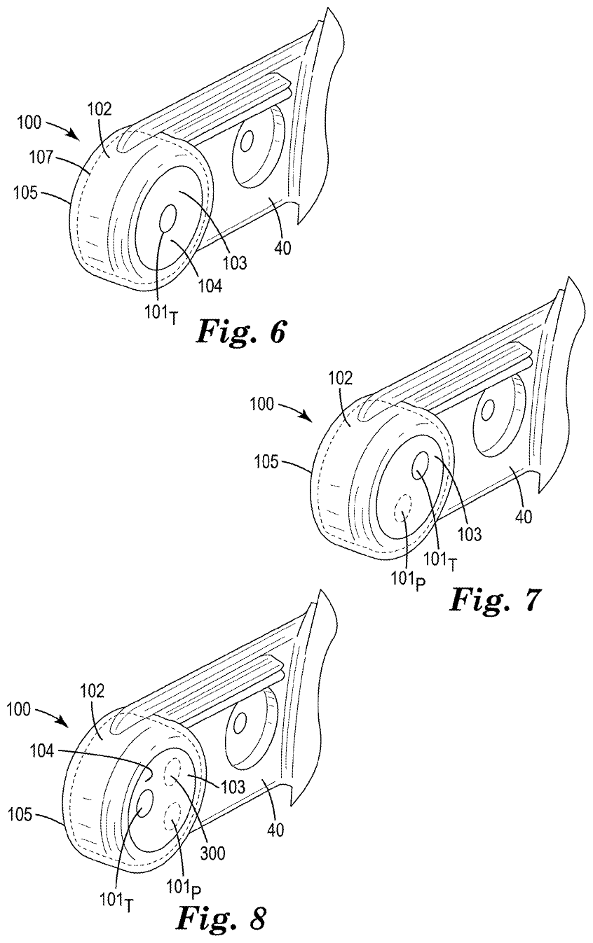

As noted, a physiological sensing unit 100 may comprise a physiological sensing unit housing 102 that comprises an inward surface 103 that comprises a contact area 104 that is configured to rest against a designated area of the wearer's head and within which at least one physiological sensor 101 is located, as evident from FIGS. 2 and 3. Any number of sensors 101, and as well as any local circuitry needed to operate such sensors, may be provided e.g. in a compartment 107 within housing 102 as indicated in FIG. 6, which is an isolated magnified view of an exemplary sensing unit 100 and its associated housing 102. (The exemplary sensing unit 100 of FIG. 6 is of the general type depicted in FIG. 4). In some embodiments a working surface of a physiological sensor 101 may be exposed on the inward side of housing 102, e.g. by way of an aperture provided in inward surface 103 for this purpose. Thus in some such embodiments, the working surface of the sensor may be in direct contact with the user's head (e.g. with the skin of the user's head).

In some embodiments, a physiological sensor 101 (e.g., at least a working surface of the sensor) may be located behind (outward of) a portion of an inward wall of housing 102. Such arrangements may depend on the operating mechanism of the sensor; specifically, whether the operating mechanism allows the presence of an intervening layer between the working surface of the sensor and the user's head. In some instances (e.g. if the sensor is a photoplethysmographic sensor as discussed later) at least a portion of the inward wall of the sensor housing within contact area 104 may take the form of a clear “window” made of a material that is transmissive to electromagnetic radiation in the wavelength range of interest.

In some embodiments a physiological sensor 101 of a physiological sensing unit 100 may be a temperature sensor 101_{T}, as indicated in exemplary, generic illustration in FIG. 6. By this is meant a sensor that obtains data that is correlated with the body temperature of the wearer of the facemask. It is noted that such a sensor may not necessarily provide data that is in the form of an actual numerical temperature e.g. on a Centigrade scale. Rather, the sensor may provide data in the form of an electrical voltage or other signal that is then processed by sensor control module 15 to convert the data to an estimated body temperature. Such a temperature sensor may operate by any suitable mechanism; for example, it may be an infrared temperature sensor. By definition, a temperature sensor 101_{T} of a physiological sensing unit 100 as disclosed herein will be positioned within 5 mm of the skin of the user's head. Thus if the temperature sensor 101_{T} is an infrared sensor, it will operate at very close range, in contrast to infrared temperature sensors that are configured to sense temperature at distances of e.g. one or more cm. In some embodiments, such an infrared sensor may comprise a window (and/or a portion of an inward wall of housing 102 of sensing unit 100 may comprise such a window) made of a material that is transmissive to infrared radiation.

In some embodiments a physiological sensor of a physiological sensing unit 100 may be a “contact” temperature sensor. By a contact temperature sensor is meant a sensor that must be in contact with the user's head in order to function properly. In some embodiments a suitable aperture may be provided in contact area 104 of the inward wall of the sensor unit housing. This can allow the front (inward), working face of the contact temperature sensor to be flush with the inward major surface of contact area 104, or to protrude slightly inward beyond the inward major surface of the sensor housing, so that the working face of the temperature sensor can be pressed against the user's head, e.g. skin. However, as noted, in some embodiments such a sensor may be able to function even if a housing wall is present between the working face of the temperature sensor and the user's head. If present, such a housing wall should be chosen to have suitable properties, e.g. to exhibit high thermal conductivity, low heat capacity, and so on. In general, in some embodiments the sensor unit housing 102 and/or any other components that are present on or in the housing, may be configured to have a relatively low heat capacity and/or to be thermally isolated from the working face of the temperature sensor, to ensure that the housing does not act as a heat sink that unacceptably disturbs the body temperature as detected by the temperature sensor. The contact temperature sensor 101_{T} may operate by any suitable mechanism; for example, it may take the form of a thermistor, a thermocouple, or resistance temperature detector (e.g. comprising a serpentine or wire-wound metal such as platinum). Such a sensor may take any physical form, e.g. a rigid “button”, a flexible electronic circuit, and so on.

In some embodiments a physiological sensor of a physiological sensing unit 100 may be a heat flux sensor. A heat flux sensor measures the rate of heat transfer per unit area of a surface; such sensors often comprise semiconductors that can generate a voltage proportional to the passage of heat. Sensors of this general type are described e.g. in U.S. patent Ser. No. 10/088,373; some such sensors are available e.g. under the trade designation CORE from greenTEG (Zurich, Switzerland). In some embodiments, a heat flux sensor may serve as a temperature sensor. That is, a heat flux temperature may be used in combination with a suitable algorithm that manipulates the observed heat flux in order to obtain an estimate of a body temperature (e.g. a core body temperature as discussed below) of the user. In some embodiments, a heat flux sensor may serve as an adjunct to a temperature sensor (e.g. of any of the types described above). That is, a heat flux sensor may be used along with a suitable algorithm that will adjust a sensed temperature reported by a temperature sensor, in accordance with the observed heat flux.

Sensing of body temperature can be advantageous for persons that are wearing an SCBA and facemask. Often, such persons may be firefighters or first responders who are wearing personal protective equipment (such as heavy trousers, boots, jacket, and so on), and may be doing so in close proximity to fires and/or in hot weather. Such a combination of conditions can increase the possibility of a heat-related condition, syndrome or illness such as e.g. dehydration, hyperthermia, heat exhaustion, or heatstroke. Accordingly, it may be advantageous to monitor the body temperature of such a person e.g. in order to detect the possibility, onset, or presence, of a heat-related condition. In some embodiments, the temperature that is tracked, reported, etc., by the herein-disclosed physiological monitoring system will be a body temperature. This is a general term that encompasses the “sensed” temperature of the person; that is, the temperature that is reported by sensor 101_{T} (of course, the data from the sensor may need to be processed to turn the data in its raw form, into a numerical temperature value). The term body temperature also encompasses the “dermal” temperature of the person (including e.g. the epidermis and potentially at least a portion of the dermis), as obtained e.g. by taking the “sensed” temperature and correcting for any locally distorting effects (e.g., any heat-sink effect of the temperature sensor itself that might cause the “sensed” temperature to be slightly below the “dermal” temperature).

The term body temperature further encompasses a sensed or dermal temperature that has been processed by the circuitry of the physiological monitoring system to convert the temperature into a “core body temperature”. As will be well understood, the core body temperature is the temperature at or near the deep structures of the body, and is generally considered to be similar to temperatures obtained e.g. by rectal, vaginal, or internal measurements (oral measurements are generally considered to provide slightly lower values). By way of a specific example, a person might exhibit a sensed temperature of e.g. 35 degrees C., which, in the particular circumstances, is considered to correspond to an dermal temperature of 36 degrees C., which, in the particular circumstances, is considered to correspond to a core temperature of 38 degrees C. Any of these temperatures (in particular, the dermal temperature or the core body temperature) may be referred to herein as an “estimated” temperature, in view of the fact that the reported temperature may be an estimate rather than an “exact” temperature (noting that in some embodiments, an “exact” temperature may not necessarily be needed, as discussed in detail below.)

The core body temperature is often considered to be the ideal parameter to monitor e.g. for purposes of assessing the possibility of a heat-related illness. The arrangements disclosed herein encompass various methods by which a physiological monitoring system can be configured to convert a sensed temperature to an estimated core body temperature. However, it is noted that at least in some instances it may not be necessary to perform such a conversion. That is, in some cases, the possibility of a heat-related condition or illness may be gauged by monitoring the sensed temperature or a dermal temperature estimated therefrom rather than attempting to convert such a temperature to an core body temperature. Furthermore, in some cases this may be gauged by monitoring the change in the sensed temperature or dermal temperature (with or without establishing a baseline temperature as a basis of comparison). Thus, for example, if the sensed temperature of a person has increased from e.g. 35 degrees C. (e.g. at the time that the facemask was donned) to e.g. 38 degrees C., this may be taken as an indication that a heat-related illness may be possible. That is, even the person's core body temperature is not specifically known, it may be considered that the core body temperature is likely to have risen commensurately with the sensed temperature and thus an alert of a possible heat-related illness may be issued.

In relying at least partially on a change in the sensed or dermal temperature, steps can be taken to ensure that a proper initial value of the temperature are used. For example, the physiological monitoring system may be configured so that an “initial” body temperature may not be established until an initial period has passed (e.g. the first 2-3 minutes after the person dons the facemask). This can ensure that any transient effects caused by the sensing unit itself (e.g., the sensing unit causing a slight temperature drop of the user's local head area until the sensing unit has thermally equilibrated with the local head area) will have ceased. If any such phenomenon is present, the physiological monitoring system may be trained or otherwise configured to disregard or adjust the temperature data until an initial transient period of e.g. thermal equilibration of the user's head with the temperature sensor is over.

In some embodiments, the physiological monitoring system may take into account the rate of change in the body temperature. This may be done either in combination with tracking the magnitude of the change in body temperature, or instead of tracking the magnitude of the change in body temperature. That is, if the rate of change in body temperature is rapid enough, this may be noted even if the magnitude of the change in body temperature is still rather small. For example, if the body temperature is observed to increase by e.g. 2 degrees C. in a few minutes, the system may flag this as a concern, issue an alert, or take other appropriate action. In short, a physiological monitoring system as disclosed herein can take appropriate action based on the body temperature increasing by a predetermined amount and/or increasing at a rate that is above a predetermined rate.

A physiological monitoring system can be configured to issue an alert signal upon temperature data being processed to provide an indication that such an alert may be appropriate. Such an alert signal may take any form. For example, it may include an audible, haptic, or visual signal that is communicated to the wearer of the facemask. However, since it is possible for a heat-related condition such as heatstroke to cause a person to be disoriented or confused, in some embodiments it may be appropriate that an alert signal be sent to someone other than the wearer of the facemask. (This may be done instead of, or in addition to, sending an alert signal to the wearer.) Thus in some embodiments, an alert signal may be a signal that is sent wirelessly to a remote device (meaning a device that is not resident on the SCBA facemask or any part of the SCBA, and that is not worn by the user of the SCBA or carried by the user on their person). In some embodiments, such a remote device may be a portable device that is carried by a designated person e.g. of a firefighting or first-response unit. In various embodiments, such a remote device may be e.g. a general purpose smartphone, tablet, or laptop (with the alert taking the form of e.g. a pop-up notification broadcast by an app resident on the remote device). Or, the remote device might be a specialized, dedicated electronic device.

In some embodiments, such a remote device may be at a central monitoring facility rather than carried by an on-scene person. Whatever the specific arrangement, in some embodiments multiple SCBA facemasks may each be monitored by way of a mask-resident physiological monitoring system, with the results being sent from each such system to a common remote device. In such a case, a common remote device may monitor multiple persons, e.g. an entire firefighting squad, company, etc. The notifications received regarding such persons may cause, for example, one or more persons to be rotated out of active firefighting for rest and/or hydration. It is emphasized that an alert signal does not necessarily have to be indicative of an ongoing or incipient medical emergency (such as e.g. heatstroke). Rather, in some embodiments a physiological monitoring system as disclosed herein may be used e.g. to determine that a particular individual is becoming overheated to a degree that is not necessarily immediately dangerous but that nevertheless may make it desirable to rotate the person out for a period of rest or hydration.

An alert signal (whether delivered to the wearer of the facemask, or to a remote device) can take any suitable form. In various embodiments, more than one kind of alert signal may be sent, e.g. of varying urgency. An alert signal may or may not include numerical information as to the body temperature of the person in question. For example, in some embodiments an alert signal may take the form of a nonquantitative (e.g. green/yellow/red) visual indicator. In some embodiments an alert signal may include information such as “estimated body temperature is X degrees C.” (whether alone or in combination with a nonquantitative indicator).

An alert signal as described above is a subset of a signal that is indicative of body temperature (including estimated body temperature). In general, a mask-resident physiological monitoring system may send any such signal to a remote device. Thus in some instances, the physiological monitoring system could send estimated body temperature values, or even raw data, to a remote device. The remote device could then perform any further data processing operations to determine whether an alert signal needs to be issued. However, in many embodiments it may be advantageous that data processing be performed by the physiological monitoring system that is resident on the facemask. This way, even if there is a momentary communication outage between the mask-resident monitoring system and the remote device, the mask-resident monitoring system would still be able to issue a local alert signal (e.g. in the form of an audible or visual signal) to alert the user of a potential heat-related illness or condition.

As noted above, in some embodiments a facemask-resident physiological monitoring system may function by sensing a temperature and may take action upon the sensed temperature changing by a certain amount and/or at a certain rate. In some embodiments, a physiological monitoring system may be trained to establish a baseline temperature for the wearer of the facemask. Such a baseline-establishing session may be somewhat similar to the previously-described procedure of determining an accurate “initial” temperature upon initially donning a facemask, in order to detect any deviation from that temperature. However, a baseline-establishing procedure may be more comprehensive.

For example, in a baseline-establishing training session, an SCBA facemask may be donned by a user and then worn by the user under “baseline” conditions. By this is meant that the user is not engaged in heavy exertion and is not under stressful conditions (for example, this may be done in a firehouse, at near-room temperature, and without heavy exertion). During this time, the monitoring system can record temperature data that is used to establish an estimated baseline body temperature of the user. This baseline temperature may then be taken into account later, in actual-use conditions. For example, an alert signal may be issued if the estimated body temperature of the user differs from the estimated baseline body temperature by a predetermined amount.

Such an arrangement takes into account more than simply a change in temperature from an initial temperature during a single period of wearing the facemask. Rather, it takes into account any difference from, and/or change away from, and/or rate of change away from, a baseline temperature of the user as was established over one or more training periods of wearing the facemask. Such a baseline temperature may be established by training the physiological monitoring system in any number of training sessions. In some embodiments, such sessions may be performed at various times of day and so on. In such embodiments, the physiological monitoring system may learn and store a baseline body temperature that, rather than being constant, takes into account e.g. the variation in that particular user's body temperature according to the user's circadian rhythm. Still further, in some embodiments the system may be able to use additional data as accumulated in actual use of the facemask (e.g., in fire situations rather than in firehouse training sessions) to assemble more complete picture of the user's body temperature, how it varies daily, how it vanes with environmental conditions, and so on. This can allow the system to more accurately assess whether a particular set of temperature data, in a given circumstance, is indicative of a possible heat-related illness or condition.

As noted earlier, in some embodiments it may be desired to use the temperature data acquired by the one or more temperature sensors 101_{T} to provide an estimate of the user's core body temperature. In such embodiments, the system may be configured to correlate a sensed temperature with a core body temperature. A relatively straightforward example would be to perform one or more training sessions, e.g. in a variety of conditions, at different times of day, etc., and to take measurements that closely approximate the person's core body temperature (e.g. by way of oral or other temperature measurements). In this way any characteristic difference or offset between the core body temperature (as best approximated by available measuring techniques) and the temperature sensed by the mask-resident physiological monitoring system, can be determined. Then, in future, the system can use this knowledge to convert a sensed temperature to an estimated core body temperature. Depending upon the comprehensiveness of the training sessions, any such offset can be determined as a function of time of day, ambient conditions, and so on. Such training can be used to convert a baseline body temperature to a baseline core body temperature, if desired. In establishing such parameters, any suitable mathematical model or quantitative algorithm(s) may be used to relate a sensed temperature to an estimated core body temperature.

Thus in summary, the arrangements disclosed herein can use temperature data in a variety of ways, ranging e.g. from using real-time sensed data essentially as-is, to combining such data with historical data that provides a baseline temperature for comparison, to converting such data to an estimated core body temperature of the user. Any of these approaches, in any combination, may be used.

In some embodiments, a physiological sensor 101 of a physiological sensing unit 100 may be a photoplethysmographic sensor 101p, as indicated in exemplary, generic illustration in FIG. 6. In the depicted embodiment of FIG. 6, the photoplethysmographic sensor is present along with an above-described temperature sensor 101_{T}; however, in some embodiments, a photoplethysmographic sensor may be the only physiological sensor that is present. By a photoplethysmographic sensor is meant a sensor that optically detects any change in fluid (e.g. blood) volume in a microvascular bed of tissue. Such a photoplethysmographic sensor may comprise one or more LEDs (emitting e.g. red or green light) and one or more detectors to detect reflected light. A facemask-resident photoplethysmographic sensor that is abutted against the person's head may operate in reflectance mode (rather than in transmissive mode as is commonly done in pulse oximetry using a photoplethysmographic sensor that is mounted on a translucent body part such as a fingertip). However, in some embodiments a mask-resident photoplethysmographic sensor may be contacted with the wearer's ear and thus may function in transmissive mode.

Photoplethysmographic sensors are most commonly known for use in measuring oxygen saturation. A mask-resident photoplethysmographic sensor as disclosed herein may be used for such a purpose. However, in many embodiments, such a photoplethysmographic sensor may be used to monitor other parameters. For example, since the beating of the heart causes blood to slightly distend the arteries and arterioles of the subcutaneous tissue of the head, a photoplethysmographic sensor may be used to monitor the heart rate (pulse) of the person. Since the heart rate may be affected by a heat-related illness such as e.g. heatstroke, this may provide useful data that can be used in a determination of e.g. whether to issue an alert signal. In some embodiments, a physiological monitoring system may use the previously-described body temperature data and heart rate data in combination, for such a purpose.

In some embodiments a mask-resident photoplethysmographic sensor may be able to monitor more detailed parameters of the mask-wearer's heart function, for example at least some parameters, features or behaviors of the wearer's cardiac cycle. These may include e.g. detection of possible tachycardia, fibrillation, or premature ventricular contractions (PVCs). Although such phenomena are often detected by electrical methods (EKGs), in some instances they may be detected by photoplethysmography since the pulsatile component of the cardiac cycle may cause variation in the blood volume in the subcutaneous tissue that is sufficiently observable that such features can be detected. In some embodiments, the respiration rate (e.g. in breaths per minute) of the user may be monitored, since respiration can cause fluid-perfusion effects that are superimposed on the cardiac cycle and that may be detectable by a photoplethysmographic sensor under at least some conditions. In some embodiments, a photoplethysmographic sensor may be able to monitor whether the user is in a hypovolemic state. (The photoplethysmographic sensor may also be able to monitor whether the user is in a hypervolemic state, but detection of hypovolemia may be more useful e.g. in monitoring for heat-related conditions or illnesses.) In some embodiments, a photoplethysmographic sensor may be able to detect erythema; that is, reddening of the skin, which may likewise be useful in monitoring for heat-related conditions or illnesses.

It will be appreciated that any of these parameters, as monitored by a photoplethysmographic sensor, may be helpful e.g. in detecting a potential, or existing, heat-related illness or condition (of course, any such parameter may be useful for other purposes as well). Thus in various embodiments, a physiological sensing unit may comprise a photoplethysmographic sensor configured to detect one or more of these parameters, or may comprise multiple photoplethysmographic sensors that are configured to detect various parameters. In various embodiments, any of these parameters, or any combination of these parameters, may be used in addition to monitoring temperature as discussed earlier.

While FIG. 7 depicts an exemplary arrangement in which a temperature sensor 101_{T} and a photoplethysmographic sensor 101p are disposed rather closely together within the same housing, this does not have to be the case. In particular, if the accuracy of the temperature sensing might be affected by the thermal mass of a housing that is large enough to hold two (or more) sensors, the temperature sensor may be disposed in a separate housing. In a variation of this approach, a bifurcated (e.g., Y-shaped) housing may be used with the temperature sensor disposed at the end of one leg of the Y and a photoplethysmographic sensor disposed at the end of the other leg. Such arrangements may minimize any heat-sink effect that may be caused by the sensor housing in the local vicinity of the temperature sensor.

Beyond the above uses, a photoplethysmographic sensor may serve still another function (or, in some cases, it may be present primarily to serve this other function). Specifically, in some embodiments the data that is obtained by the photoplethysmographic sensor may assist in determining whether a contact-temperature sensor is pressed against the user's head with sufficient force that the temperature data can be considered to be reliable. For example, if the intensities of reflections that are collected by a photodetector of the photoplethysmographic sensor fall below a certain value, it may be inferred that the sensing unit housing may have been displaced or disturbed, so that the temperature data received during that time may be considered to be questionable. The physiological monitoring system may take this into account in any suitable way (for example, it may simply delete the questionable temperature data from the data stream; and/or, it may notify the wearer of the facemask to check the status or physical positioning of the sensor housing(s)).

In some embodiments, an SCBA facemask that comprises a mask-resident physiological monitoring system may also comprise a bone conduction communication system. As shown in exemplary embodiment in FIG. 8, such a system may comprise at least one bone conduction transducer 300 that is configured to be pressed against the mask-wearer's head so as to be able to broadcast sound waves into the skull and therefrom to the inner ear. (The term skull is used in general, and encompasses the temporal bone, the mastoid bone, the zygomatic bone, the sphenoid bone, the maxilla, and so on.) In such an arrangement neither transducer 300 nor any other component of the bone conduction system penetrates through or into the skin of the wearer; such a communication system thus differs from a bone-anchored hearing aid or a cochlear implant. In the depicted embodiment of FIG. 8, the bone conduction transducer 300 is disposed in the same housing as a physiological sensor (in the depicted embodiment, two such sensors are present, a temperature sensor 101_{T} and a photoplethysmographic sensor 101p). However, in some embodiments, the bone conduction transducer may be located in a separate housing or at an end of a bifurcated or branched housing of the general type mentioned earlier herein. (Such a housing may have any number of branches that are needed according to the number of transducers and/or sensors.) Such arrangements may be appropriate if the temperature sensor needs to be thermally isolated from the other sensors and/or transducers, and/or if the emission of sound waves by the bone conduction transducer may interfere with the functioning of the physiological sensor(s) in some manner.

In some embodiments, all such transducers and sensors, whether co-located closely together in the same housing (e.g. as in FIG. 8), or spaced apart from each other at ends of a branched housing, or spaced apart from each other in separate housings, may be configured so that tightening a strap coupling element of the facemask will cause all of the transducers and sensors to be pressed against the user's head. Of course, the sensors may be configured so that each transducer or sensor experiences a pressing force appropriate to facilitate proper operation of the transducer or sensor.

A bone conduction communication system may include any suitable type of bone conduction transducer 300. In many embodiments, such a system will include (at least) two such transducers, e.g. one for each side of the person's head, to provide for binaural performance. Such a bone conduction communication system can advantageously allow the person to receive e.g. radio communications while not blocking the person's ear(s) e.g. with an earbud, thus allowing the person to wear hearing protection devices (e.g. earplugs) while still receiving radio communication.

A bone conduction communication system may comprise any appropriate circuitry that is needed to facilitate the functioning of the system. Such circuitry may be disposed e.g. in an electronic control unit 11 of the general type described earlier herein, or in a sensor control module 15 (e.g. along with circuitry that facilitates the operation of the physiological sensors). In some embodiments, various portions or subsets of the circuitry for operating the bone conduction communication system may be disposed in both locations or in some other location of the facemask. In some embodiments, the bone conduction communication system may be used for inbound communications. In such instances, the facemask may handle outbound communications by way of a conventional microphone (mounted somewhere in the interior of the facemask) that picks up voice utterances by the mask-wearer and transforms them into electrical signals for broadcasting in any suitable manner.

Bone conduction communication systems, arrangements of bone conduction transducers, and details of circuitry that may be used to operation such systems, are described in detail in U.S. provisional patent application 62/376,113, in the resulting PCT application published as WO 2018/035207, and in the resulting U.S. patent application Ser. No. 16/326,002, all of which are incorporated by reference herein in their entirety.

An SCBA facemask as disclosed herein may comprise any other sensor for any suitable purpose. For example, in some embodiments the SCBA facemask may comprise at least one temperature sensor that is located on the outside of the facemask to provide an indication of the temperature in the environment in which the person is located. In some embodiments the SCBA facemask may comprise at least one temperature sensor that is located on the inside of the facemask so as to provide an indication of the temperature in the previously-mentioned interior volume 3 of the facemask. In some embodiments the SCBA facemask may comprise an accelerometer, e.g. to allow the movements of the person to be tracked so that an indication can be obtained of the person's physical exertions. Any or all such information may be taken into account by the physiological monitoring system (e.g. as an adjunct to the body temperature), in determining e.g. whether an issuance of an alert signal may be appropriate.

In some embodiments a housing 102 of a sensing unit 100 may comprise one or more ancillary items that may enhance the performance of a physiological sensor 101 (and/or of a bone conduction transducer 300). For example, in some embodiments an absorbent material and/or a fluid-wicking material may be disposed on the inward side of the housing of a sensing unit, e.g. on the periphery of the housing. Such an arrangement may minimize the chance of perspiration penetrating into the space between the sensor and the user's head; or, it may allow any perspiration that develops between the user's head and the sensor to be wicked away.

In some embodiments a physiological sensor need not necessarily be attached to, or connected to, or even relatively close to, a strap coupling element, as long as the sensor is positioned and configured so that donning the facemask and snugging the mask and head harness tight, causes the sensor to be pressed against the user's head to an appropriate degree. For example, in some embodiments, a physiological sensor might be incorporated into a face seal 4, or into a nosecup 7. Any such physiological sensor, however arranged and supported, can be configured so that donning the facemask presses the sensor against an area of the user's head that is suitable for allowing the sensor to operate. In some embodiments, such an area might be generally in front of the auricle, generally to the rear of the auricle, vertically even with the ear canal, generally above the ear canal, generally below the ear canal, and so on. If multiple sensors (and e.g. bone conduction transducers) are present, they may each be placed in the most appropriate position for that entity. In some embodiments, multiple sensors of the same type (e.g. multiple temperature sensors) may be present.

Although discussions herein have primarily concerned use of SCBA facemasks by firefighters or first responders, the arrangements disclosed herein may be used in any circumstance in which it is appropriate for a person to wear an SCBA facemask. Such circumstances are not necessarily limited to those in which fire or smoke is present. Rather, the presence or potential presence of any hazardous airborne entity may cause a need for an SCBA facemask. Thus, an SCBA facemask as disclosed herein may be suitable for e.g. military uses, law enforcement uses, medical uses (whether in a hospital or in the field) and so on. Such a facemask may also find use in industrial settings, e.g. for inspection of tanks or other enclosures that potentially harbor dangerous gases or other airborne substances, and so on. Such an SCBA facemask need not necessarily be worn in combination with e.g. firefighter apparel, but rather may find use in other circumstances, e.g. when a person is wearing a hazmat suit.

The herein-disclosed arrangements may serve as an addition to procedures and safeguards already existing for e.g. firefighters and first responders. Such arrangements may, in some instances, provide an enhanced ability to detect a potential heat-related illness or condition at least slightly earlier than may otherwise occur. Such arrangements cannot provide a guarantee of detecting or preventing any possible condition. In particular, the arrangements disclosed herein, and the characterizations of such arrangements herein, will not be interpreted as allowing a wearer of an SCBA facemask to depart from established procedures or to ignore any warning signs or symptoms of a possible heat-related illness or other medical condition.

It will be apparent to those skilled in the art that the specific exemplary embodiments, elements, structures, features, details, arrangements, configurations, etc., that are disclosed herein can be modified and/or combined in numerous ways. Numerous variations and combinations are contemplated as being within the bounds of the conceived invention, not merely those representative designs that were chosen to serve as exemplary illustrations. Thus, the scope of the present invention should not be limited to the specific illustrative structures described herein, but rather extends at least to the structures described by the language of the claims, and the equivalents of those structures. Any of the elements that are positively recited in this specification may be explicitly included in the claims or excluded from the claims, in any combination as desired. Any of the elements or combinations of elements that are recited in this specification in open-ended language (e.g., comprise and derivatives thereof), are considered to additionally be recited in closed-ended language (e.g., consist and derivatives thereof) and in partially closed-ended language (e.g., consist essentially, and derivatives thereof). To the extent that there is any conflict or discrepancy between this specification as written and the disclosure in any document that is incorporated by reference herein, this specification as written will control.

What is claimed is:

1. A self-contained breathing apparatus (SCBA) facemask with a physiological monitoring system, comprising:
a facemask configured to be worn by a user, the facemask defining an interior region adjacent the user's face when the facemask is donned by the user;
a physiological monitoring system that is resident on the facemask and that comprises an integrated physiological sensing unit that is attached to the facemask and that is configured to obtain data correlated with a physiological parameter of the user of the facemask by way of a physiological sensor that is mounted in a housing of the physiological sensing unit within a contact area of an inward side of the housing of the physiological sensing unit,
wherein the facemask and the physiological sensing unit are configured so that upon the facemask being donned by the user, the contact area of the housing of the physiological sensing unit is pressed against the head of the user so that the physiological sensor is in position to obtain the data correlated with the physiological parameter of the user of the facemask.

2. The SCBA facemask of claim 1 wherein the physiological sensor comprises a contact temperature sensor.

3. The SCBA facemask of claim 2 wherein the physiological sensing system comprises a sensor control unit that receives temperature data from the contact temperature sensor and that is configured to use the temperature data to obtain an estimate of a body temperature of the user and to issue an alert signal if the estimated body temperature increases by a predetermined amount and/or if the estimated body temperature increases at a rate that is above a predetermined rate.

4. The SCBA facemask of claim 3 wherein the sensor control unit of the physiological sensing system is configured to convert the estimated body temperature of the user to an estimated core body temperature of the user and to issue an alert signal if the estimated core body temperature increases by a predetermined amount and/or if the estimated body temperature increases at a rate that is above a predetermined rate.

5. The SCBA facemask of claim 3 wherein the sensor control unit of the physiological sensing system comprises a wireless transmitter that is configured to wirelessly transmit an alert signal to a remote device based on the estimated body temperature.

6. The SCBA facemask of claim 1 wherein the physiological sensing sensor comprises a photoplethysmographic sensor.

7. The SCBA facemask of claim 6 wherein the physiological sensing system of the SCBA facemask comprises a sensor control unit that receives photoplethysmographic data from the photoplethysmographic sensor and that is configured to use the photoplethysmographic data to monitor at least one of heart rate and respiration of the user.

8. (canceled)

9. The SCBA facemask of claim 1 wherein the integrated physiological sensing unit comprises a first physiological sensor configured to obtain data correlated with a first physiological parameter of the user of the facemask, and a second physiological sensor configured to obtain data correlated with a second physiological parameter of the user of the facemask, the second physiological parameter being different from the first physiological parameter.

10. The SCBA facemask of claim 9 wherein the first physiological sensor is a contact temperature sensor and wherein the second physiological sensor is a photoplethysmographic sensor.

11. (canceled)

12. The SCBA facemask of claim 1 wherein the SCBA facemask further comprises an integrated bone conduction communication unit that comprises a bone conduction transmitter that is configured so that upon the facemask being donned by the user, the physiological sensor and the bone conduction transmitter are both pressed against the head of the user so that the physiological sensor is in position to obtain data correlated with a first physiological parameter of the user and the bone conduction transmitter is in position to perform bone conduction transmission of sound waves to an inner ear of the user.

13. The SCBA facemask of claim 12 wherein the bone conduction transmitter is mounted in the housing of the physiological sensing unit, within the contact area of the inward side of the housing of the physiological sensing unit.

14. The SCBA facemask of claim 1 wherein the housing of the physiological sensing unit is attached to a strap coupling element of the facemask so that when the strap coupling element is connected to a strap of a head harness and snugged tight in the process of donning the facemask, the contact area of the housing is pressed against the head of the user so that the physiological sensor is in position to obtain the data correlated with the physiological parameter of the user of the facemask.

15. The SCBA facemask of claim 14 wherein the strap coupling element is an upper strap coupling element that integrally extends from a face seal of the SCBA facemask.

16. The SCBA facemask of claim 1 wherein the facemask and the physiological sensing unit are configured so that upon the facemask being donned by the user, the contact area of the housing of the physiological sensing unit is pressed against the head of the user so that the physiological sensor is in contact with an area of the head of the user proximate a superficial temporal artery or a branch thereof.

17. A self-contained breathing apparatus (SCBA) comprising the SCBA facemask of claim 1 and a supply of breathing air that is fluidly connected to the facemask.

18. A method of performing physiological monitoring of a user of an SCBA facemask and communicating sound to the user of the SCBA facemask, and the method comprising:
donning an SCBA facemask that is equipped with a facemask-resident physiological sensing system comprising an integrated physiological sensing unit, the facemask being further equipped with an integrated bone conduction communication unit that comprises a bone conduction transmitter,
wherein the donning of the facemask causes a contact surface of a housing of the integrated physiological sensing unit to be pressed against a head of the user and causes the integrated bone conduction transmitter to be pressed against the head of the user,
and,
after the facemask has been donned, repeatedly performing all of the following actions, sequentially and/or concurrently, in any order:
obtaining temperature data from a contact temperature sensor that is mounted in the housing of the integrated physiological sensing unit,
obtaining photoplethysmographic data from a photoplethysmographic sensor that is mounted in the housing of the integrated physiological sensing unit,
and,
transmitting sound to the inner ear of the user by way of the bone conduction transmitter.

19. The method of claim 18 wherein the temperature data is sent from the contact temperature sensor to a sensor control unit of the facemask-resident physiological sensing system, wherein the temperature data is processed by the sensor control unit to provide an estimate of the user's body temperature, and wherein the method further comprises issuing an alert signal if the estimated body temperature increases by a predetermined amount or if the estimated body temperature increases at a rate that is above a predetermined rate.

20. The method of claim 19 wherein the alert signal comprises an alert signal that is wirelessly transmitted from the sensor control unit to a remote device that is not resident on the SCBA facemask and is not worn by, or carried on the person of, the user of the SCBA facemask.

21. The method of claim 20 wherein a plurality of estimated body temperatures are obtained from a plurality of SCBA facemasks that are each equipped with a facemask-resident physiological sensing system, and wherein a signal indicative of each estimated body temperature is wirelessly transmitted from a sensor control unit of each physiological sensing system of each facemask, to a common remote device that receives all such signals.

22. The method of claim 19 wherein the method comprises one or more preliminary steps of donning the SCBA facemask and wearing the facemask for a period of time under nominal conditions during which temperature data is obtained by the contact temperature sensor and processed by the sensor control unit to provide an estimate of the user's baseline body temperature; and wherein in subsequent use of the SCBA facemask, the method comprises issuing an alert signal if the estimated body temperature of the user differs from the estimated baseline body temperature of the user by a predetermined amount.
